(19) United States
(12) Patent Application Publication
(10) Pub. No.: US 2024/0268768 A1
(43) Pub. Date: Aug. 15, 2024

(54) Method of Preventing or Treating Pancreatic Dysfunction or Diabetes by Upregulating Human Cathelicidin LL-37 to Inhibit Islet Amyloid Polypeptide (IAPP) Self-Assembly

(71) Applicant: The Board of Trustees of the Leland Stanford Junior University, Stanford, CA (US)

(72) Inventors: Valentina Armiento, Kranzberg (DE); Annelise E. Barron, Woodside, CA (US); Aphrodite Kapurniotu, Muenich (DE); John A. Fortkort, Austin, TX (US)

(73) Assignee: The Board of Trustees of the Leland Stanford Junior University, Stanford, CA (US)

(21) Appl. No.: 17/759,710

(22) PCT Filed: Jan. 28, 2021
(86) PCT No.: PCT/US2021/015531

§ 371 Date: Jul. 28, 2022

Related U.S. Application Data
(60) Provisional application No. 62/967,023, filed on Jan. 28, 2020.

(51) Int. Cl. A61B 5/00; A61B 5/145; A61K 31/164; G06T 7/00

(52) U.S. Cl. CPC A61B 5/7275; A61B 5/14532; A61B 5/14546; A61K 31/164; G06T 7/0012

(57) ABSTRACT

A method for treating type 2 diabetes (T2D) is provided. The method comprises diagnosing a subject as suffering from T2D or as being pre-diabetic; monitoring the response to glucose stimulation of at least one islet in the pancreas of the subject by quantitatively imaging glucose metabolism in vivo; establishing a target range for the response to glucose stimulation of the at least one islet; and upregulating cathelicidin gene expression in the subject until the monitored response to glucose stimulation is within the target range.

### CROSS-REFERENCE TO RELATED APPLICATION

This application claims the benefit of U.S. provisional application No. 62/967,023, filed Jan. 28, 2020, having the same title, and having the same inventors, and which is incorporated herein by reference in its entirety.

### SEQUENCE LISTING

This application includes material in an electronic Sequence Listing filed concurrently herewith. The material in the electronic Sequence Listing is submitted as a text (.txt) file entitled “07148PCT_SeqList_ST25.txt” created on Jan. 27, 2021, which has a file size of approximately 7 KB. The electronic Sequence Listing is hereby incorporated by reference in its entirety.

### FIELD OF THE DISCLOSURE

The present disclosure relates generally to methods for treating diseases of the pancreas (such as type 2 diabetes), and more particularly to methods for modulating cathelicidin gene expression for the treatment of such diseases.

### BACKGROUND OF THE DISCLOSURE

Amyloid self-assembly of islet amyloid polypeptide (IAPP) is linked to pancreatic β-cell degeneration and the pathogenesis of type 2 diabetes (T2D). The 37-residue IAPP is secreted from the β-cells together with insulin, and acts in its soluble form as a neuropeptide regulator of glucose homeostasis. However, under conditions of T2D, the intrinsically disordered but highly amyloidogenic IAPP self-assembles into cytotoxic oligomers and amyloid fibrils, which mediate pancreatic inflammation and β-cell degeneration.

### SUMMARY OF THE DISCLOSURE

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In one aspect, a method is provided for preventing or treating type 2 diabetes (T2D). The method comprises (a) diagnosing a subject as suffering from T2D or as being pre-diabetic; (b) monitoring the response to glucose stimulation of at least one islet in the pancreas of the subject by quantitatively imaging glucose metabolism in vivo; (c) establishing a target range for the response to glucose stimulation of the at least one islet; and (d) upregulating cathelicidin gene expression in the subject until the monitored response to glucose stimulation is within the target range.

In another aspect, a method is provided for treating a subject for type 2 diabetes (T2D). The method comprises (a) diagnosing the subject as suffering from T2D; and (b) applying to the subject a pharmaceutically acceptable composition which upregulates cathelicidin gene expression in the subject.

In another aspect, a method is provided for treating a prediabetic subject. The method comprises (a) diagnosing the subject as pre-diabetic or likely to suffer from T2D in the future; and (b) applying to the subject a pharmaceutically acceptable composition which upregulates cathelicidin gene expression in the subject.

In a further aspect, a method is provided for treating islet amyloid polypeptide (IAPP) aggregation and accumulation in the pancreas of a subject. The method comprises (a) detecting the presence of IAPP aggregate accumulation in pancreatic tissues of the subject; and (b) administering to the subject a pharmaceutically acceptable composition which upregulates cathelicidin gene expression in the pancreatic tissues of the subject, and/or systemically.

In another aspect, a method is provided for treating a subject. The method comprises (a) monitoring levels of the cathelicidin peptide LL-37 in the blood of a subject and IAPP amyloid in pancreatic tissues of the subject; and (b) when the condition L/B<k is detected, where L is the level of LL-37 detected, B is the level of IAPP detected, and k is a predetermined threshold value, upregulating cathelicidin gene expression in the subject.

In still another aspect, a method is provided for modulating in vivo IAPP amyloid fibril formation. The method comprises (a) monitoring the level of IAPP amyloid in pancreatic tissues of a subject; and (b) administering to the subject a pharmaceutically active composition which modulates in vivo fibril formation in said pancreatic tissues by inducing the expression of a physiologically effective binding partner for IAPP amyloid.

In yet another aspect, a method is provided for modulating in vivo fibril formation in the pancreatic tissues of a subject. The method comprises (a) co-incubating IAPP amyloid with a physiologically effective binding partner for IAPP amyloid, thereby obtaining co-incubated peptides; (b) creating a pharmaceutical composition from the co-incubated polypeptides; and (c) administering the pharmaceutical composition to a subject.

In still another aspect, a method is provided for inhibiting in vivo IAPP amyloid fibril formation in the pancreatic tissues of a subject in which an equilibrium exists between smaller and larger MW species of IAPP amyloid. The method comprises administering to the subject a pharmaceutical composition which shifts the equilibrium toward the smaller species of IAPP amyloid.

In still another aspect, a method is provided for preventing the formation of IAPP fibrils and plaques in the pancreas by inducing the pancreatic and/or systemic expression of LL-37, which is encoded by the human CAMP gene.

In yet another aspect, a method is provided for treating a subject for type 2 diabetes (T2D). The method comprises (a) diagnosing the subject as suffering from T2D; and (b) applying to the subject a pharmaceutically acceptable composition comprising a peptidomimetic of LL-37 or a portion thereof.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIGS. 1-5 illustrate the effects of LL-37 on IAPP amyloid self-assembly and cell-damaging effects. Thus, FIG. 1 depicts the fibrillogenesis of IAPP (16.5 μM) alone or with LL-37 (1/1) determined by ThT binding (means (±SD), 3 assays); LL-37 alone is shown for comparison (1 assay).

FIG. 2 is a series of TEM images of solutions from 1a (7 days aged) as indicated (bars, 100 nm); inset in LL-37 image shows LL-37 fibrils (minor population).

FIG. 3 is a graph of cell viability of cultured RIN5fm cells after treatment with IAPP and its mixtures from 1a (7 days aged) determined by MTT reduction (means (±SD), 3 assays (n=3 each)); effects of LL-37 alone are also shown (1 assay, n=3).

FIG. 4 depicts a graph of the IC50 of inhibitory effect of LL-37 on IAPP cytotoxicity determined by titration of IAPP (100 nM; red symbol) with LL-37 and MTT reduction (means (±SD), 3 titration assays (n=3 each)).

FIG. 5 is a graph of the fibrillogenesis of IAPP (16.5 μM) alone or with LL-37 (1/1) following seeding with fIAPP (10%) determined by ThT binding (means (±SD), 3 assays).

FIGS. 6-11 provide characterization of the LL-37-IAPP interaction.

FIG. 6 is a graph depicting the determination of the app. Kd by fluorescence spectroscopic titrations. Fluorescence emission spectra of Fluos-IAPP (5 nM) alone or with various amounts of LL-37 (pH 7.4) as indicated. Inset, binding curve (means (±SD), 3 titration assays).

FIG. 7 is a graph and associated photomicrograph depicting the binding of FAM-LL-37 to IAPP monomers and fibrils as determined by DB. IAPP monomers and fibrils (40 μg) were spotted on a nitrocellulose membrane and probed with FAM-LL-37 (200 nM) (results representative of 4 assays).

FIG. 8 is a Far-UV CD spectra of IAPP (5 μM), IAPP-LL-37 (1/1; 5 μM each), and LL-37 (5 μM) (0 h) (pH 7.4); the sum of the spectra of LL-37 and IAPP is also shown.

FIGS. 9-10 are kinetic follow-ups of IAPP misfolding alone (FIG. 9) or with LL-37 via far-UV CD spectroscopy. Spectra of IAPP (FIG. 9) and its 1/1 mixture with LL-37 (FIG. 10) at various time points are shown (conditions as in FIG. 8).

FIG. 11 is a characterization of IAPP-LL-37 hetero-assemblies via cross-linking (pH 7.4), NuPAGE, and WB (IAPP 30 μM; IAPP/LL-37, 1/0.1 or 1/1). A representative gel (n>5) is shown.

FIGS. 12-13 depict the LL-37 binding to IAPP fibrils (fIAPP) converts them into seeding incompetent assemblies.

FIG. 12 is a graph depicting the fibrillogenesis of IAPP (16.5 μM) alone or following seeding with 10% fIAPP or with 10% LL-37-treated fIAPP determined by ThT binding (means (±SD), 3 assays).

FIG. 13 is a series of TEM images of solutions from 3a: fIAPP seeds, LL-37-treated fIAPP seeds, and IAPP seeded with fIAPP (10%) (red dot) or LL-37-treated fIAPP (10%) (blue dot) (both at 6 h); bars, 100 nm.

FIGS. 14-16 illustrate the identification of LL-37 regions mediating its interaction with IAPP and its potent amyloid inhibitor function.

FIG. 14 is a graph depicting the fibrillogenesis of IAPP (16.5 μM) alone or in the presence of LL-37(1-14) or LL-37(15-37) (1/1) as determined by ThT binding (means (±SD), 3 assays).

FIG. 15 is a graph depicting the β-cell-damaging effects of solutions from 4a (24 h aged) determined by MTT reduction (RIN5fm cells) (means (±SD), 3 assays (n=3 each)).

FIG. 16 is an identification of LL-37 regions that bind IAPP using peptide microarrays. Glass slides with decamers consisting of overlapping LL-37 sequences (bold) were incubated with Fluos-IAPP (1 μM); visualization by fluorescence. Identified IAPP binding clusters in dashed blue line frames; LL-37 “binding cores”, red letters (results representative from 4 assays).

FIG. 17 is an illustration of the suggested protective role of LL-37-IAPP interaction in pancreatic amyloid formation, inflammation, β-cell degeneration, and T2D pathogenenesis.

FIG. 18 is a graph showing the dose-dependence of the inhibitory effect of LL-37 on IAPP fibrillogenesis: Fibrillogenesis of IAPP (16.5 μM) alone or with different molar ratios of LL-37 as indicated was determined by the ThT binding assay (means (+SD), 3 assays).

FIGS. 19-22 depict the effects of scrLL-37 on IAPP fibrillogenesis, cell-damaging effects, and conformation.

FIG. 19 depicts fibrillogenesis of IAPP (16.5 μM) alone or with scrLL-37 (IAPP/scrLL-37, 1/10) determined by the ThT binding assay (means (±SD), 3 assays).

FIG. 20 depicts the effects on IAPP cytotoxicity: Solutions from S2a (7 days aged) were added to RIN5fm cells and cell damage was assessed by MTT reduction (means (±SD), 3 assays (n=3 each)).

FIG. 21 is a series of TEM images of solutions (7 days aged) from S2a of IAPP alone and its mixture with scrLL-37; bars, 100 nm). In the inset of the TEM image of the mixture, amorphous aggregates, found to be in addition to the fibrils a major aggregate population and most likely corresponding to scrLL-37 (10-fold excess), are shown.

FIG. 22 depicts the effects of scrLL-37 on IAPP conformation studied by far-UV CD spectroscopy: CD spectra of IAPP (5 μM), the mixture of IAPP with scrLL-37 (1/1; 5 μM each), and scrLL-37 (5 μM) (pH 7.4) are shown; for comparison, the sum of the spectra of scrLL-37 and IAPP is also shown.

FIG. 23 is a characterization of LL-37 homo-oligomers (including homo-tetramers at ˜15 kDa) via cross-linking with glutaraldehyde, NuPAGE, and WB with anti-LL-37 antibody: LL-37 was incubated (30 min) at concentrations of 3 and 30 μM corresponding to IAPP/LL-37 ratios of 1/0.1 or 1/1 alone or in the presence of IAPP (30 μM; gel shown in FIG. 2f) (pH 7.4). The blot shown is representative of 3 experiments.

FIG. 24 is a set of β-amyloid imaging probes which may be utilized to image pancreatic islets.

FIG. 25 depicts the primary structures of IAPP, LL-37, scrambled LL-37 (scrLL-37), and LL-37 segments synthesized and studied (IAPP has a C-terminal amide; LL-37 and related peptides have a C-terminal COOH). IAPP and LL-37 sequence alignment was performed by LALIGN.

### DETAILED DESCRIPTION

Turning to the data and figures, when taken together along with the instant disclosure provide, according to many embodiments, methods for treating type 2 diabetes (T2D). Many such embodiments include monitoring the response to glucose stimulation of at least one islet in the pancreas, establishing a target range for the response to glucose stimulation of the at least one islet; and upregulating cathelicidin gene expression in the subject until the monitored response to glucose stimulation is within the target range. Various embodiments are also directed to methods for treating a subject. Many such embodiments include monitoring levels of the cathelicidin peptide LL-37 in the blood of a subject and IAPP amyloid in pancreatic tissues of the subject. Still many embodiments are directed to methods for treating a subject for type 2 diabetes (T2D) including applying to the subject a pharmaceutically acceptable composition comprising a peptidomimetic of LL-37 or a portion thereof.

Type 2 diabetes is characterized by insufficient levels of insulin secretion from pancreatic islet 0-cells to compensate for insulin resistance. Whether a patient with insulin resistance progresses to overt diabetes depends on islet β-cell dysfunction. In order to track the progression of the disease and to understand how various risk factors (such as, for example, obesity) affect proper islet function, it is necessary to have a technique for assessing islet function in vivo. To date, blood insulin measurements are frequently used for this purpose, but such measurements are typically insensitive to small changes, and may be affected by other physiological activities.

Embodiments according to the current disclosure establish that the foregoing issues may be overcome through the use of an imaging platform that combines intrinsic autofluorescence with multiphoton excitation microscopy. This platform provides a noninvasive means for obtaining high-resolution images which may be utilized to quantitatively image real-time glucose metabolism from single islets in vivo, while also offering the advantages of greater imaging depths (and, in some applications, reduced photobleaching and reduced photodamage). Consequently, this technique provides the ability to repeatedly measure an islet's response to glucose stimulation in real time, while also providing the ability to simultaneously monitor islet function, proliferation, vasculature and macrophage infiltration in vivo from a single set of images.

Sun et al. (Sun J, Furio L, Mecheri R, van der Does A M, Lundeberg E, Saveanu L, Chen Y, van Endert P, Agerberth B, Diana J. Pancreatic β-Cells Limit Autoimmune Diabetes via an Immunoregulatory Antimicrobial Peptide Expressed under the Influence of the Gut Microbiota. Immunity. 2015 Aug. 18; 43(2):304-17. doi: 10.1016/j.immuni.2015.07.013. Epub 2015 Aug. 4. PMID: 26253786, the disclosure of which is incorporated by reference) found that antimicrobial peptides (AMPs) expressed by epithelial and immune cells are largely described for the defense against invading microorganisms. Recently, their immunomodulatory functions have been highlighted in various contexts. However, the manner by which AMPs expressed by non-immune cells might influence autoimmune responses in peripheral tissues (such as the pancreas) is unknown.

It has been found that insulin-secreting β-cells produce the cathelicidin related antimicrobial peptide (CRAMP) and that this production is defective in non-obese diabetic (NOD) mice. CRAMP administrated to prediabetic NOD mice induced regulatory immune cells in the pancreatic islets, dampening the incidence of autoimmune diabetes. Additional investigation revealed that the production of CRAMP by β-cells was controlled by short-chain fatty acids produced by the gut microbiota. Accordingly, gut microbiota manipulations in NOD mice modulated CRAMP production and inflammation in the pancreatic islets, revealing that the gut microbiota directly shape the pancreatic immune environment and autoimmune diabetes development.

Amyloid self-assembly of islet amyloid polypeptide (IAPP) is linked to pancreatic inflammation, β-cell degeneration, and the pathogenesis of type 2 diabetes (T2D). The multifunctional host defense peptides (HDPs) cathelicidins play crucial roles in inflammation. It has now been found that the antimicrobial and immunomodulatory polypeptide human cathelicidin LL-37 binds IAPP with nanomolar affinity and effectively suppresses its amyloid self-assembly and related pancreatic β-cell damage in vitro. In addition, key LL-37 segments have been identified which mediate its interaction with IAPP. The foregoing suggests a possible protective role for LL-37 in T2D pathogenesis and offers a molecular basis for the design of LL-37-derived peptides combining antimicrobial, immunomodulatory, and T2D-related anti-amyloid functions as promising candidates for multifunctional drugs.

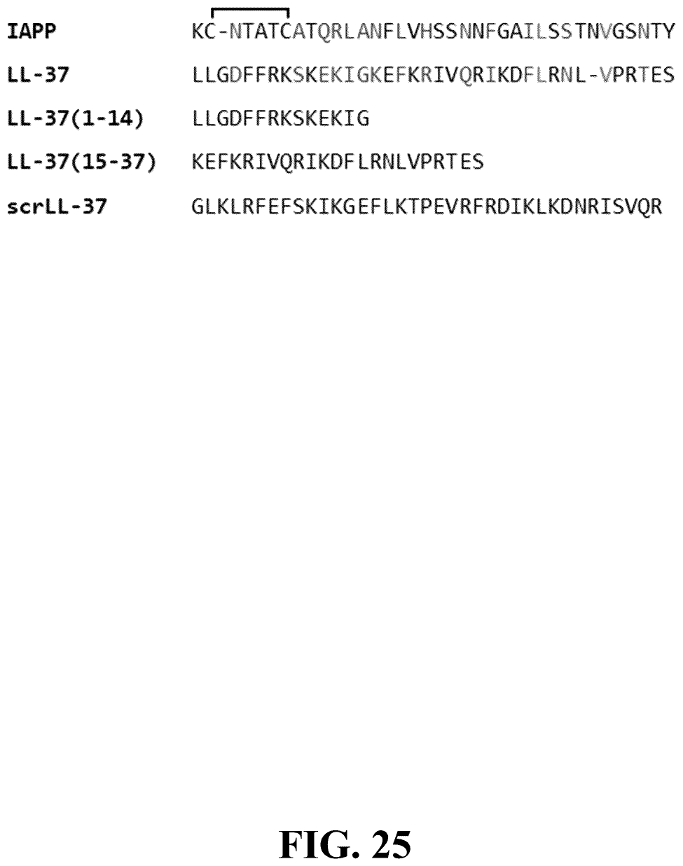

The multifunctional host defense peptides (HDPs) cathelicidins play crucial roles in inflammatory processes. This includes both pro-inflammatory and anti-inflammatory roles. So far, the only known human cathelicidin is LL-37 (FIG. 25). LL-37 is a 37-residue polypeptide which is broadly expressed by a plethora of immune and non-immune cells, including the β-cells of the pancreas. LL-37 plays a crucial role in innate immunity. Its best known functions are its broad-spectrum antimicrobial activity and its potent immunomodulatory effects. Importantly, secretion of the mouse LL-37 orthologue cathelicidin related antimicrobial peptide (CRAMP) by pancreatic β-cells was recently found to suppress pancreatic β-cell inflammation in a mouse model of type 1 diabetes (T1D) by converting inflammatory cells into regulatory ones. In addition, CRAMP/LL-37 treatment promoted insulin and glucagon secretion and enhanced islet function. Thus, a protective role for LL-37 in T1D has been suggested. The multifunctional nature of LL-37 makes it of high biomedical importance, and numerous studies toward the design of LL-37-derived peptides with antimicrobial or immunomodulatory functions have been reported.

Increasing evidence suggests that interactions of amyloidogenic polypeptides with other polypeptides are crucial modulators of amyloid self-assembly. For instance, high affinity interactions of non-fibrillar species of IAPP with insulin or amyloid β peptide (Aβ40(42)) of Alzheimer's disease (AD) have been found to suppress IAPP amyloidogenesis in vitro. In addition, LL-37 was recently shown to interact with Aβ42, resulting in suppression of Aβ42 amyloidogenesis and neuroinflammation in vitro.

The current disclosure establishes that LL-37 also interacts with IAPP. Notably, LL-37 and IAPP share a remarkable (42%) sequence similarity (Scheme 1). The current disclosure further establishes that LL-37 binds with nanomolar affinity to IAPP and effectively suppresses its amyloid self-assembly and related pancreatic β-cell-damage in vitro. In addition, key LL-37 segments are identified which mediate its interaction with IAPP.

TABLE 1 depicts the primary structures of IAPP, LL-37, scrambled LL-37 (scrLL-37), and LL-37 segments synthesized and studied (IAPP, C-terminal amide; LL-37 and related peptides, C-terminal COOH). IAPP and LL-37 sequence alignment was performed by LALIGN.

TABLE 1
Key LL-37 segments mediating its interaction with IAPP

| | Primary Structure | SEQ ID |
|---|---|---|
| IAPP | 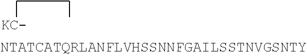 | 1 |
| LL-37 | LLGDFFRKSKEKIGKEFKRIVQRIKDFLRNL- | 2 |
| | VPRTES | |
| LL-37 (1-14) | LLGDFFRKSKEKIG | 3 |
| LL-37 (15-37) | KEFKRIVQRIKDFLRNLVPRTES | 4 |
| scrLL-37 | GLKLRFEFSKIKGEFLKTPEVRFRDIKLKDNRISVQR | 5 |

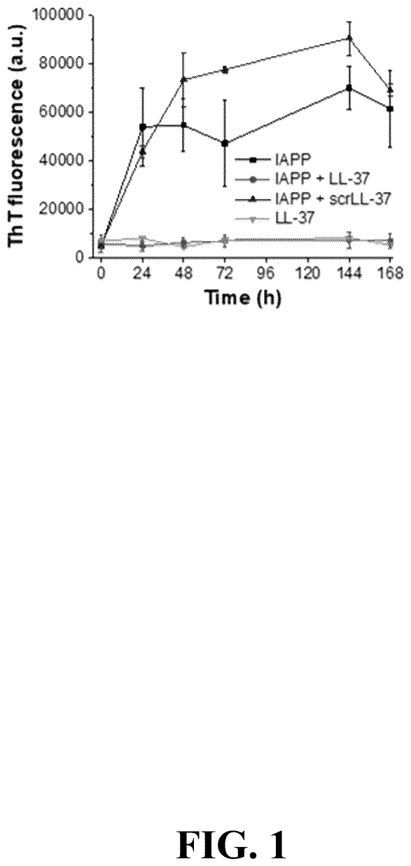

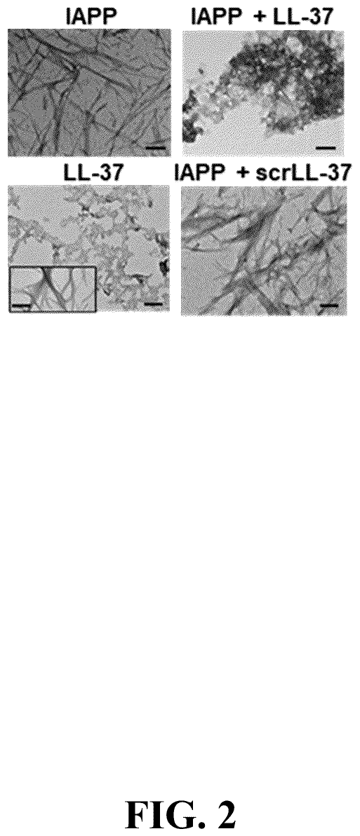

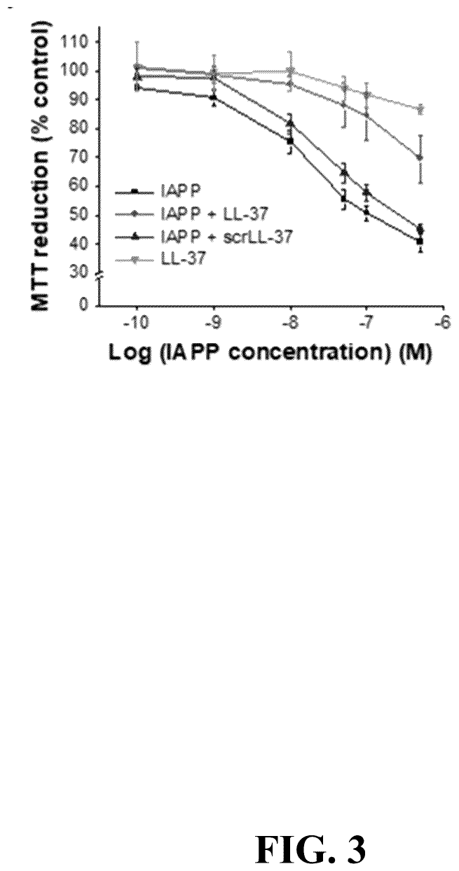

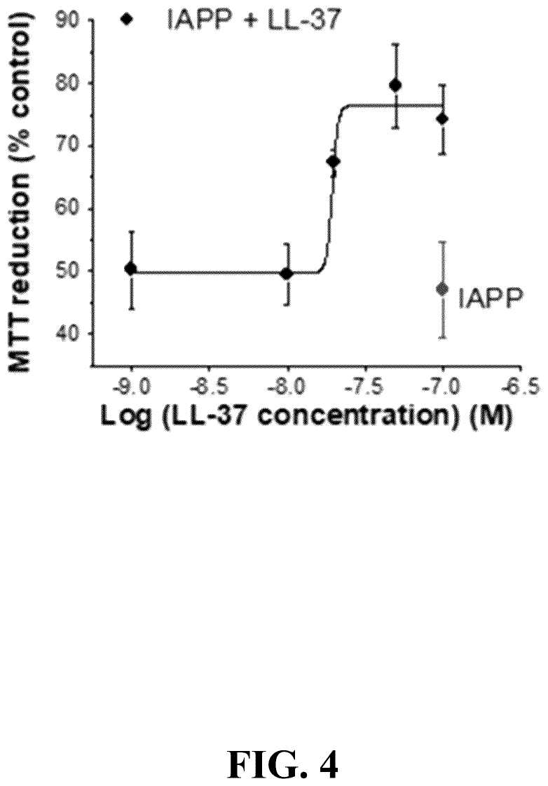

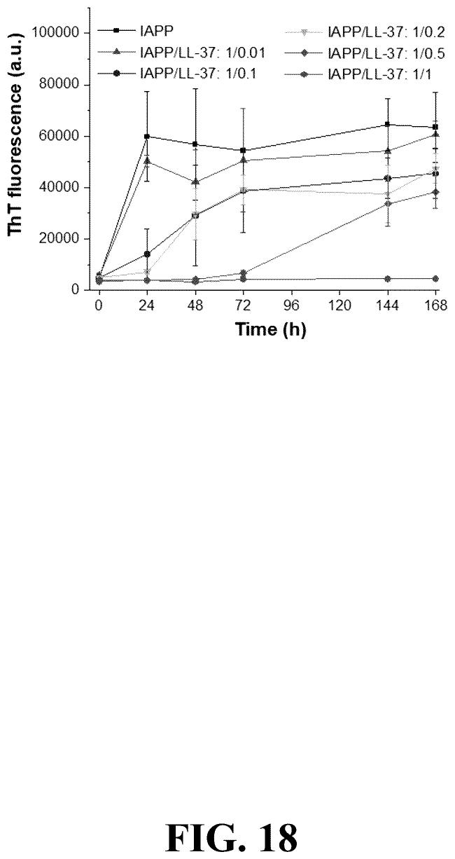

The question was first addressed whether LL-37 might interfere with IAPP amyloidogenesis and formation of cell-damaging assemblies by using the ThT binding assay in combination with TEM and a cell viability assay (FIGS. 1-5). In fact, LL-37 (1/1) effectively suppressed IAPP amyloid self-assembly (FIG. 1). The results of the ThT assay were confirmed by TEM, which revealed amorphous aggregates as major species in aged IAPP-LL-37 mixtures (FIG. 2). Interestingly, a few fibrils were also observed in aged LL-37 alone in addition to amorphous aggregates consistent with previous findings. The dose-dependence of the amyloid inhibitory effect was confirmed by additional studies (FIG. 18). Addition of the above solutions to cultured pancreatic β-cells (RIN5fm) and determination of cell damage via the 3-[4,5-dimethylthiazol-2-yl]-2,5-diphenyltetrazolium bromide (MTT) reduction assay showed that LL-37 effectively suppressed formation of cytotoxic IAPP assemblies as well (FIGS. 3-4). Of note, scrambled LL-37 (scrLL-37) was unable to inhibit up to an at least 10-fold molar excess and LL-37 alone was not cytotoxic (Scheme 1, FIGS. 1-3 & 19).

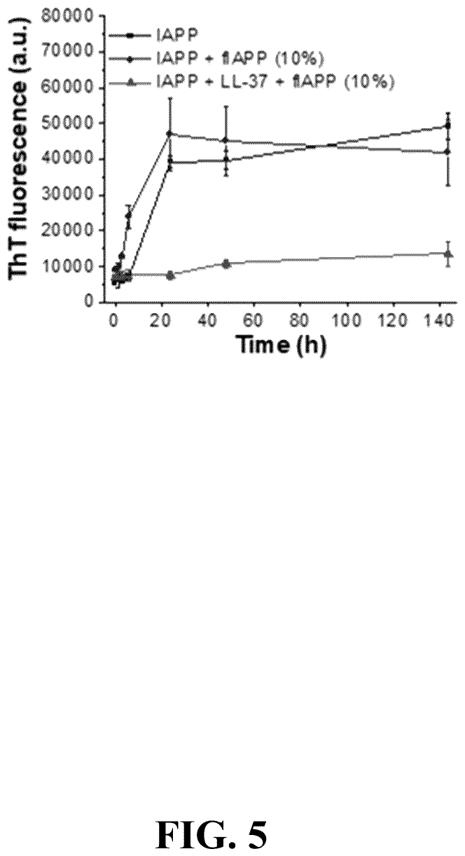

To quantify the inhibitory activity of LL-37, titrations of cytotoxic IAPP with LL-37 were performed and an IC50 of 17 (+1.7) nM was obtained (FIG. 4); thus, LL-37 is a nanomolar inhibitor of IAPP cytotoxic self-assembly. Furthermore, it was investigated whether LL-37 may also interfere with nucleation of IAPP fibrillogenesis by addition of seed amounts of preformed IAPP fibrils (fIAPP). In fact, in the presence of LL-37 (1/1), the seeding effect of fIAPP (10%) was fully suppressed (FIG. 5).

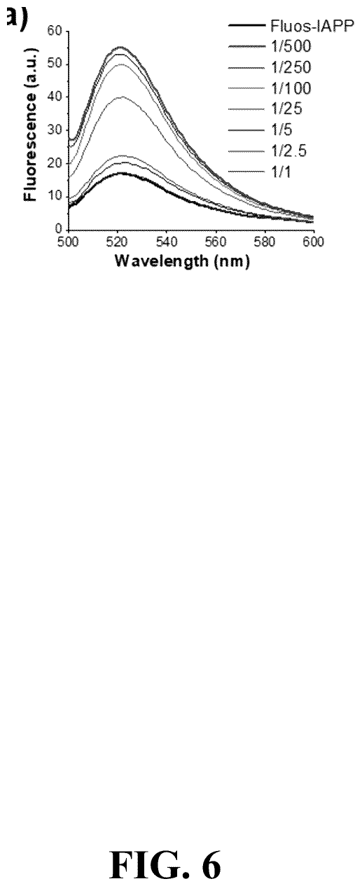

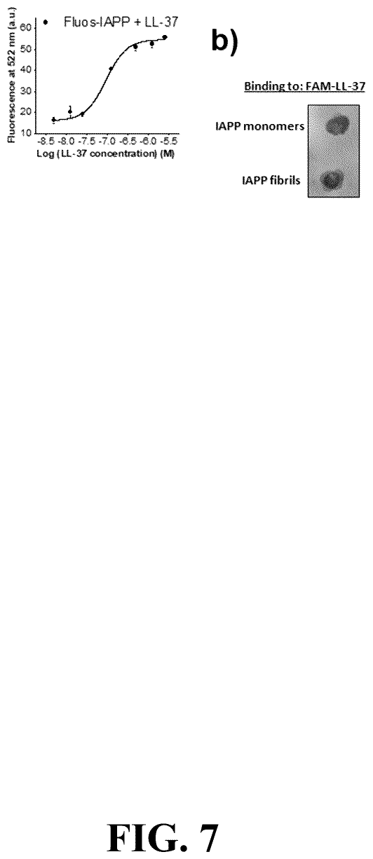

To characterize the LL-37-IAPP interaction, fluorescence spectroscopic titrations, CD spectroscopy, cross-linking, and dot blots (DBs) were performed. First, titration of N-terminal fluorescein labeled IAPP (Fluos-IAPP; 5 nM) with various amounts of LL-37 was performed. Its interaction with 100-fold molar excess of LL-37 resulted in a 322% increase of its fluorescence emission (FIG. 6). The titration yielded an apparent (app.) Kd of 88.1 (+12) nM consistent with a high affinity interaction (FIG. 6). As freshly made solutions of Fluos-IAPP at 5 nM consist mainly of monomers, these results suggested that LL-37 binds monomeric IAPP with nanomolar affinity. To find out whether LL-37 binds IAPP fibrils as well, DBs were performed using N-terminal fluorescein-labeled LL-37 (FAM-LL-37). In fact, FAM-LL-37 bound both IAPP fibrils and monomers (FIG. 7).

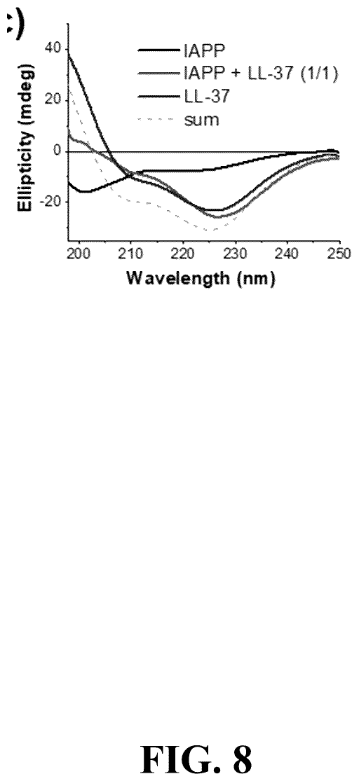

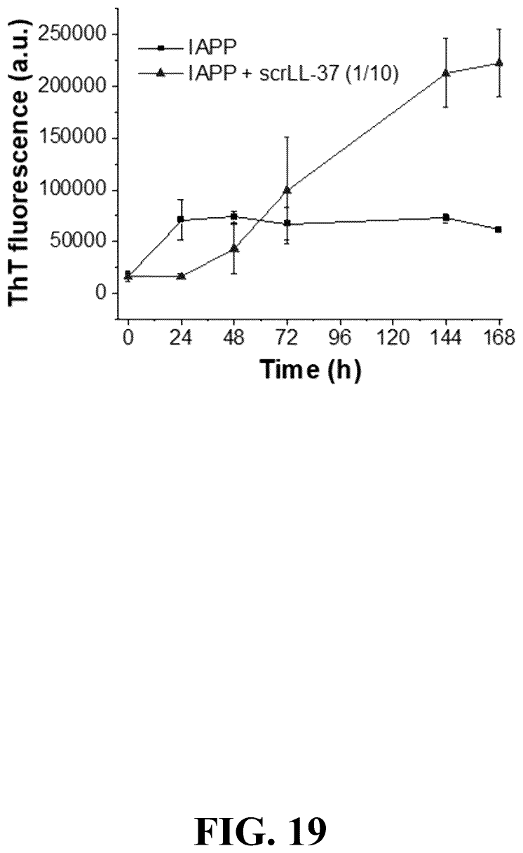

To determine the effects of LL-37 on IAPP conformation and misfolding, far-UV CD spectra of IAPP, LL-37, and the IAPP-LL-37 mixture (1/1) were measured at various incubation time points (FIGS. 8-10). The spectrum of IAPP (0 h) exhibited a strong minimum at ˜200 nm indicative of large amounts of unordered structure (FIG. 8). By contrast, the spectrum of LL-37 exhibited a strong n→π* minimum at ˜227 nm, a smaller one at ˜210 nm, and a maximum at ˜198 nm. These features were indicative of large amounts of α-helix and/or β-sheet/turn structure. Importantly, the spectrum of the mixture differed from the sum of the spectra, thus confirming the interaction between the two peptides (FIG. 8). In addition, the CD spectra of the mixture and of LL-37 were very similar to each other; α-helical homo- or hetero-oligomers could account for their 227 and 210 nm minima (FIG. 8). In fact, LL-37 has a well-known propensity to self-assemble into α-helical oligomers, while α-helix-mediated homo-dimerization may precede IAPP amyloid self-assembly. Of note, scrLL-37 (1/1) did not affect IAPP conformation (FIG. 19).

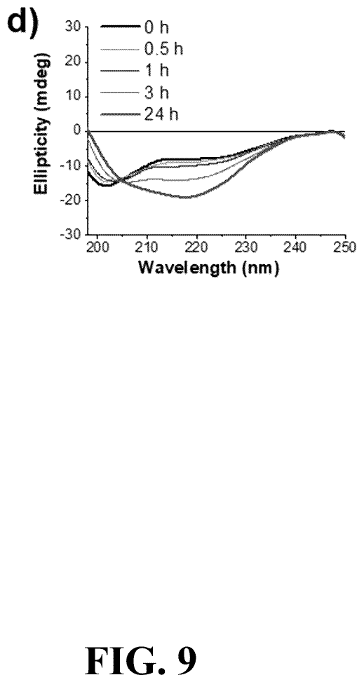

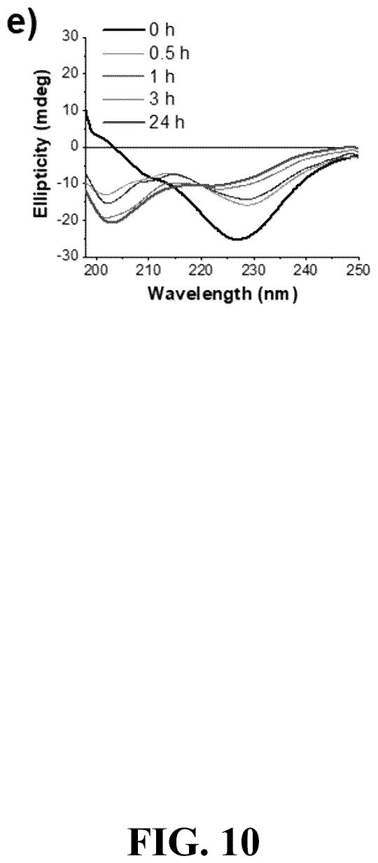

The CD spectra of IAPP at various incubation time points indicated a conformational transition into β-sheet-rich assemblies, leading to fibril formation and precipitation (24 h) (FIG. 9). By contrast, the LL-37-IAPP mixture exhibited a strong time-dependent increase of random coil contents and no precipitation occurred (FIG. 10). Thus, the LL-37-IAPP interaction resulted in soluble, partly disordered hetero-assemblies which suppressed IAPP fibrillogenesis.

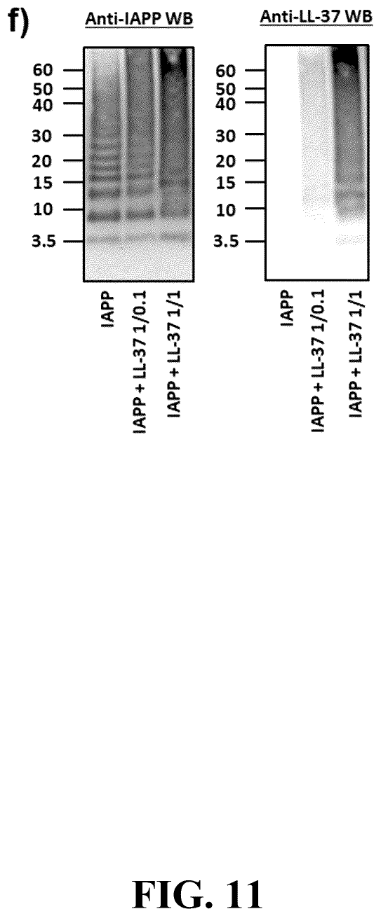

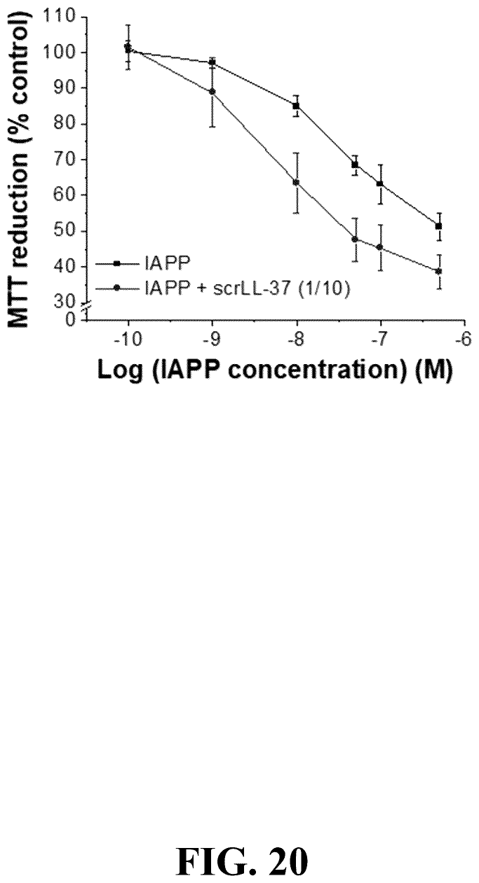

To further characterize the LL-37-IAPP hetero-assemblies, cross-linking studies were performed. LL-37-IAPP hetero-assemblies were cross-linked with glutaraldehyde, separated by NuPAGE, and visualized by Western Blot (WB) analyses with anti-IAPP and anti-LL-37 antibodies. IAPP or LL-37 alone were cross-linked as well. IAPP solutions contained low MW oligomers, mostly di- to hexamers while the smear at the upper part of the gel indicated higher MW aggregates (FIG. 11). A similar pattern was observed in the presence of non-inhibitory amounts (0.1 equivalents) of LL-37. By contrast, in the presence of an inhibitory (equimolar) LL-37 amount a novel prominent band, which was absent in the IAPP alone incubations, was found at ˜15 kDa and suggested formation of IAPP-LL-37 hetero-tetramers (FIG. 11, left panel). In addition, a strong reduction of low MW oligomeric IAPP bands likely corresponding to cytotoxic IAPP oligomers was observed (FIG. 11, left panel). WB with anti-LL-37 antibody confirmed the presence of LL-37 in the 15 kDa band of the IAPP-LL-37 mixtures (FIG. 11, right panel). Notably, LL-37 alone contained also a band at ˜15 KDa corresponding to LL-37 homo-tetramers (FIG. 20). Together, the cross-linking studies identified LL-37-IAPP hetero-tetramers as major hetero-oligomeric populations and suggested that their formation may underlie the inhibitory effect of LL-37 on IAPP amyloid self-assembly.

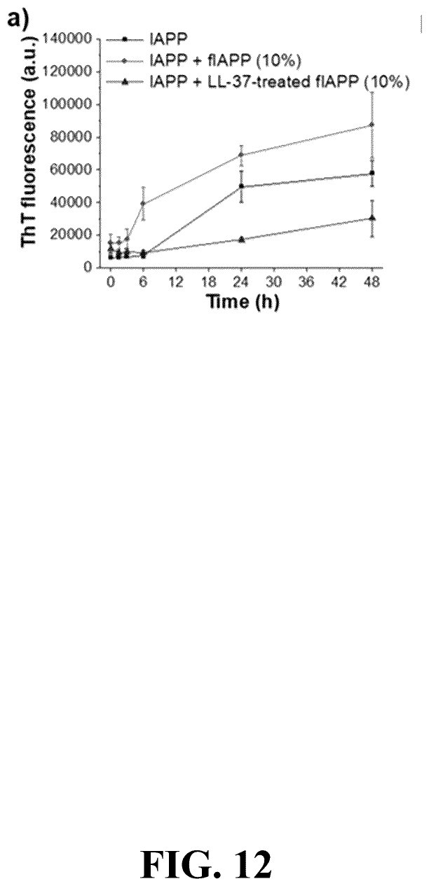

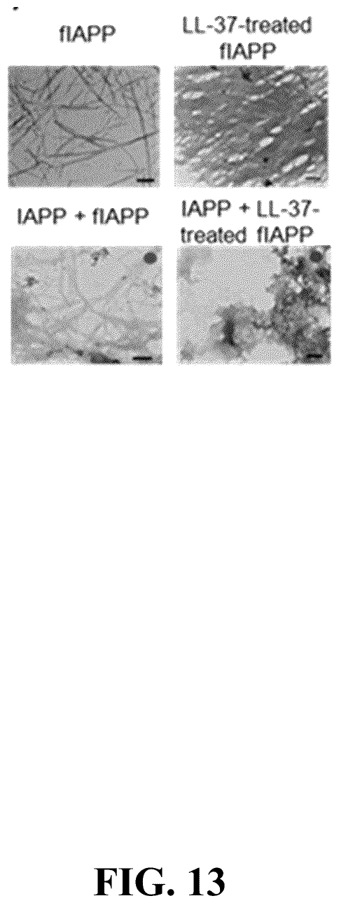

As LL-37 was found to bind to IAPP fibrils (fIAPP) as well, it was investigated whether this interaction might also contribute to its amyloid inhibitor effect. To address this, preformed fIAPP versus LL-37-treated fIAPP (i.e. fIAPP incubated with LL-37 (10-fold) for 24 h) were studied regarding their ability to act as seeds of IAPP fibrillogenesis. In contrast to untreated fIAPP (10%), which strongly accelerated IAPP fibrillogenesis, LL-37-treated fIAPP (10%) were unable to do so (FIGS. 12-13). TEM revealed marked morphological differences between fIAPP and LL-37-treated fIAPP, which stick laterally to each other into large sheet-like assemblies (FIG. 13). Thus, binding of LL-37 to IAPP fibrils converts them into seeding incompetent assemblies providing an additional mechanistic explanation for its potent amyloid inhibitor function.

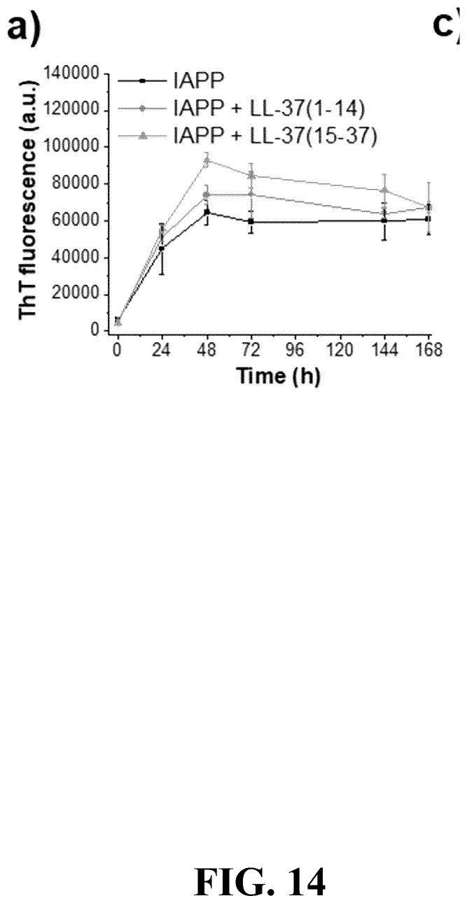

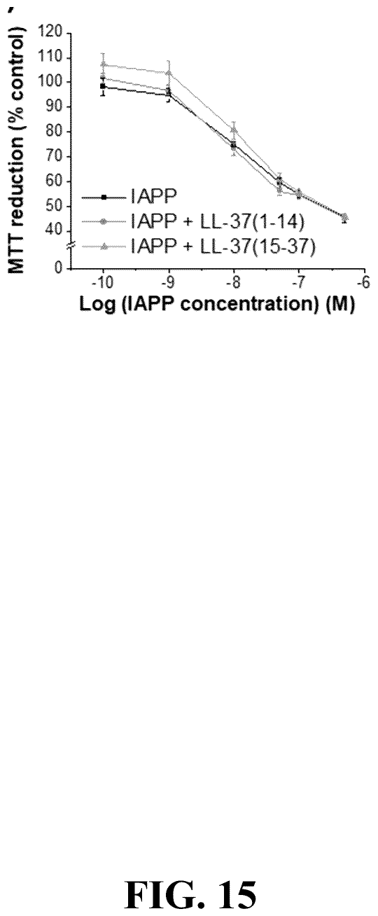

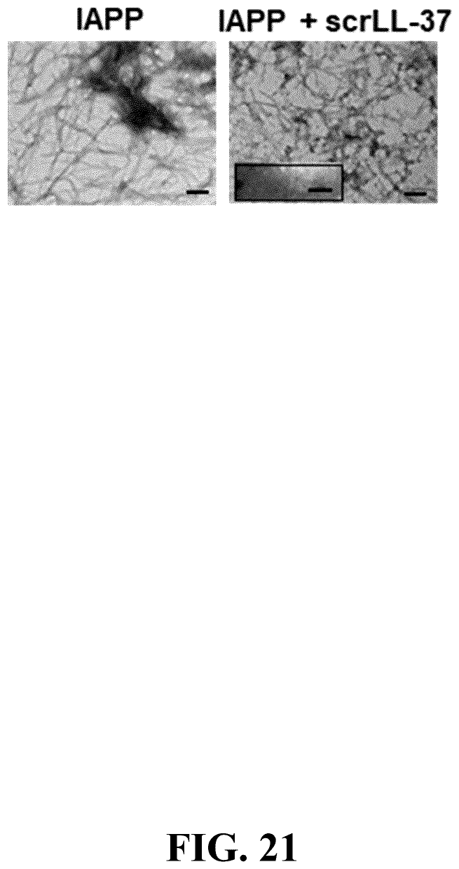

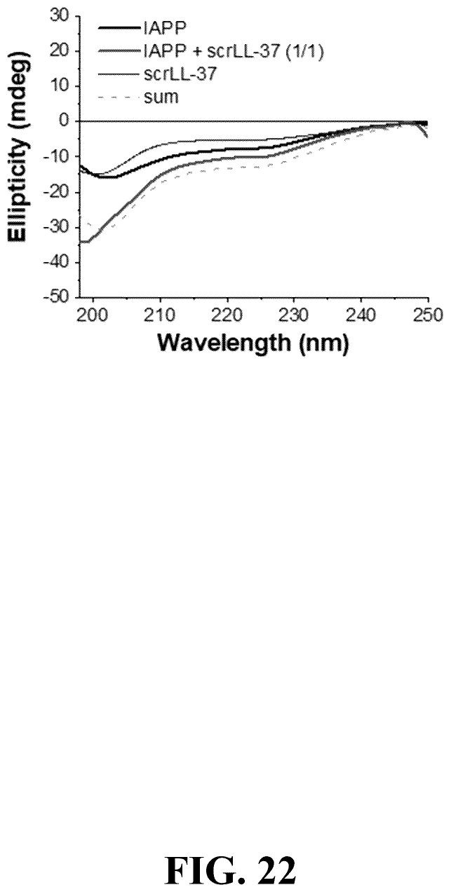

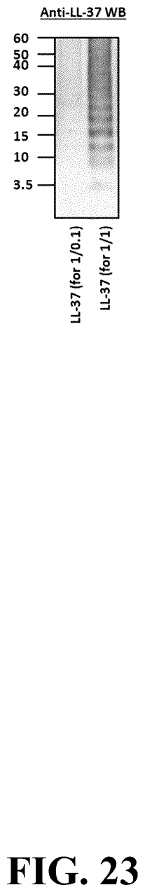

Specific partial LL-37 sequences within its central/C-terminal parts such as LL-37(17(18)-29) or LL-37(13-32) have been found to be sufficient for antibacterial, antiviral, or immunomodulatory activities and are thus being used for drug design. To find out whether the amyloid inhibitor function of LL-37 resides within specific sequence parts as well, it was dissected into the two segments LL-37(1-14) and LL-37(15-37) containing its N- and central/C-terminal helical parts. The peptides were synthesized, and their interactions and effects on IAPP amyloid self-assembly were studied. Importantly, both segments were unable to interfere with IAPP amyloid self-assembly and cell-damaging effects (1/1) (FIGS. 14-15). In addition, fluorescence titrations revealed that LL-37(15-37) bound Fluos-IAPP with high affinity (app. Kd=31.9 (+2.2) nM) as full length LL-37 as well; by contrast, a ˜30-fold weaker binding (app. Kd=2.54 (+0.5) μM) was found for LL-37(1-14) (FIG. 21). Thus, while the central/C-terminal LL-37 part likely mediates its high affinity interaction with IAPP, it is not sufficient for amyloid inhibitor function; the concerted action of central/C-terminal and N-terminal parts appears to be required.

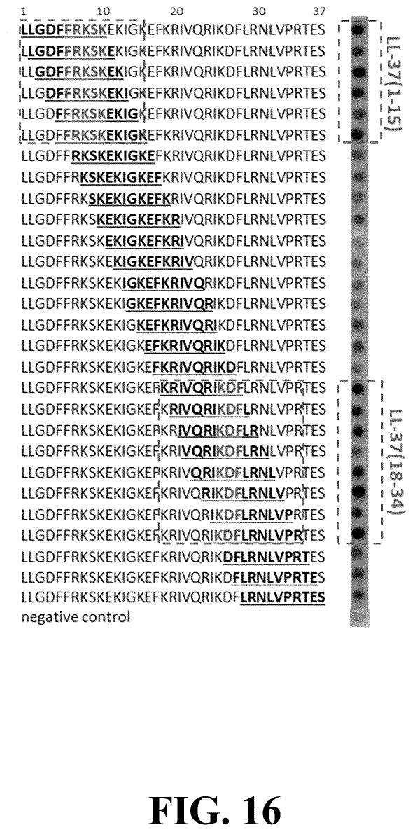

To better characterize the LL-37 regions involved in its interaction with IAPP, peptide arrays of 10-residue LL-37 segments were used covering full length LL-37 and positionally shifted by one residue; peptides were covalently attached on glass slides. Incubation with Fluos-IAPP revealed two clusters of 6-8 consecutive IAPP binding segments: the first one in the N-terminal sequence LL-37(1-15) and the second one in the C-terminal sequence LL-37(18-34) (FIG. 4c). The common sequence parts within each binding cluster (i.e., the “binding cores”) were LL-37(6-10) or FRKSK at the N-terminus, and LL-37(25-27) or KDF within the C-terminal part (FIG. 16). These findings were in line with the LL-37 dissection studies. In addition, they identified the segments mediating its interaction with IAPP.

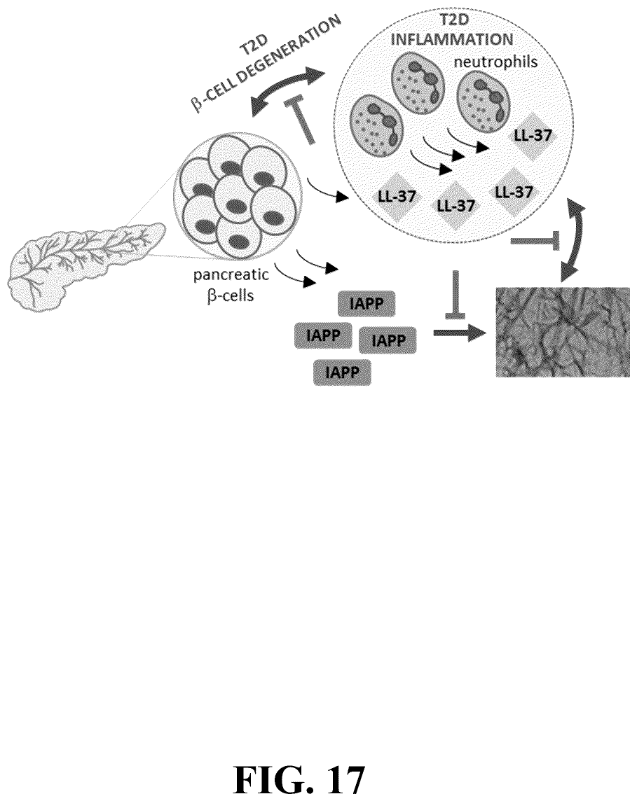

In summary, a high affinity interaction was identified between LL-37 and IAPP which effectively suppresses IAPP amyloid self-assembly in vitro, and key LL-37 segments mediate this interaction. These results suggest that LL-37 inhibitor function is mediated via its binding (a) to early prefibrillar IAPP species and their sequestration into soluble, non-fibrillar hetero-assemblies, and (b) to IAPP fibrils and their conversion into seeding incompetent assemblies. Together with findings by others, these results support the hypothesis that LL-37, secreted by pancreatic β-cells or infiltrated neutrophils under conditions of pancreatic inflammation, binds IAPP and suppresses its amyloid self-assembly and related β-cell damage, thus slowing down T2D pathogenesis (FIG. 17). Studies on the potential physiological relevance of the LL-37-IAPP interaction are now of high priority.

It will thus be appreciated that a high affinity amyloid suppressing interaction has been uncovered between a major antimicrobial and immunomodulatory polypeptide and the key amyloid polypeptide of T2D. This interaction offers a molecular basis for the design of novel molecules combining antimicrobial, immunomodulatory, and T2D-related anti-amyloid functions as candidates for multifunctional drugs.

Endogenous fluorescent cofactor NAD(P)H is a major autofluorescence signal in the cell. Because NAD(P)+ is nonfluorescent, imaging of NAD(P)H levels has been used to quantify the in situ redox state and mitochondrial function. In pancreatic islets, a method has been established to use NAD(P)H to study glucose-stimulated insulin secretion (GSIS) in vitro.

Quantitative NAD(P)H imaging of the type described herein may be utilized to directly measure glucose metabolism, and to correlate autofluorescence signals with downstream glucose-stimulated events. When glucose enters the pancreatic β-cell, NADH is first generated through glycolysis and the citric acid cycle. This phenomenon results in an increase in the ATP/ADP ratio that eventually culminates in Ca²⁺ influx and insulin secretion. The rise in cytosolic Ca²⁺ levels is followed by an increase in mitochondrial Ca² levels, which activates several dehydrogenases that form NADH in the mitochondria. This interdependence between NAD(P)H and intracellular Ca²⁺ is leveraged in some embodiments of the imaging techniques described herein to measure pancreatic β-cell function. In contrast to other in vivo imaging techniques, which typically permit monitoring of only a single aspect of islet function at a time, this approach may be utilized to simultaneously monitor multiple aspects of glucose metabolism and glucose-stimulated events from a single set of images. Thus, for example, this technique may be utilized to identify, from a single set of images, changes in glucose metabolism, islet proliferation, fibrosis, vasculature and macrophage infiltration. This is preferably accomplished through the use of multiple sources of tissue autofluorescence, which may be acquired simultaneously using multiphoton microscopy (See, e.g., Li G, Wu B, Ward M G, Chong A C, Mukherjee S, Chen S, Hao M. Multifunctional in vivo imaging of pancreatic islets during diabetes development. J Cell Sci. 2016 Jul. 15; 129(14):2865-75. doi: 10.1242/jcs.190843. Epub 2016 Jun. 6. PMID: 27270669; PMCID: PMC4958299, the disclosure of which is incorporated herein by reference).

This technique allows glucose metabolism to be imaged directly, in vivo, and in real-time in individual human islets. The technique may be applied to study human islet function at various stages during the development of diabetes, and may be utilized in conjunction with the methods disclosed herein for inducing CAMP gene expression to assess the efficacy and need for induction. Of course, while this technique is especially suitable for imaging glucose metabolism, it is also to be noted that, because many tissues in the human body share autofluorescence properties, imaging platforms of the type disclosed herein may be readily and suitably adopted to investigate or monitor a wide range of biological systems.

Various autofluorescence signals may be utilized in the devices and methodologies disclosed herein. These signals may be utilized as intrinsic biomarkers to yield detailed molecular information under both physiologic and disease states. Quantification of cellular NAD(P)H may be leveraged to monitor redox state and mitochondrial function. Cytoplasmic and mitochondrial NAD(P)H changes may be measured to resolve the spatiotemporal partitioning of glycolytic and oxidative metabolism. The distinct roles of NADH and NAD(P)H in ATP production and antioxidant defense may be resolved by simultaneously measuring two-photon NAD(P)H and one-photon lipoamide dehydrogenase autofluorescence or by fluorescence lifetime imaging. Modeling of the dynamics of lipid partitioning and fatty acid oxidation may be achieved through suitable monitoring of electron transfer flavoprotein autofluorescence. Finally, quantitative studies based on cellular autofluorescence may be utilized to distinguish between healthy and cancerous cells.

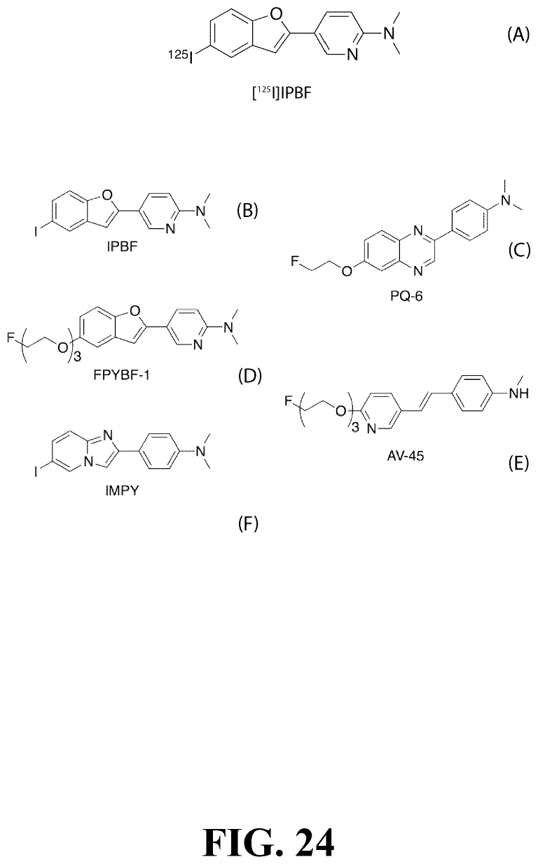

In some embodiments of the systems and methodologies disclosed herein, imaging techniques may also be employed for pancreatic islets which utilize suitable β-Amyloid imaging probes. Such probes may, for example, target amylin or other substances associated with islet amyloid deposits. These probes may be molecules that exhibit high binding affinities for A3 aggregates to allow Aβ plaques to be visualized in vivo (See, e.g., Yoshimura M, Ono M, Watanabe H, Kimura H, Saji H. Feasibility of amylin imaging in pancreatic islets with β-amyloid imaging probes. Sci Rep. 2014; 4:6155. Published 2014 Aug. 21. doi:10.1038/srep06155, the disclosure of which is incorporated herein by reference). Some particular, nonlimiting examples of probes which may be utilized for this purpose include the probes set forth in FIG. 24.

In some embodiments, positron emission tomography (PET) may be utilized in combination with amyloid imaging probes for PET detection of islet amyloid deposits. One example of such a probe is the ¹⁸F-labelled radiopharmaceutical florbetapir, which has been used elsewhere in the detection of Aβ-derived amyloid deposits in the brain for diagnosis of Alzheimer's disease. Here, it is to be noted that brain and islet amyloid deposits, though distinct, also share some structural similarities (see, e.g., Kayed R, Head E, Thompson J L, et al. (2003) Common structure of soluble amyloid oligomers implies common mechanism of pathogenesis. Science. 300(5618):486-9, the disclosure of which is incorporated herein by reference).

Various other imaging techniques and platforms may also be utilized in the systems and methodologies described herein. These include, without limitation, positron emission tomography (PET) and magnetic resonance imaging (MRI). In some embodiments, imaging techniques which utilize perfusion with Ca²⁺ probes or viral transduction of fluorescent reporters may also be utilized.

Various pharmaceutical compositions may be utilized in accordance with the teachings herein to upregulate cathelicidin gene expression in the subject, to induce the expression of a physiologically effective binding partner for IAPP amyloid, to inhibit in vivo IAPP amyloid fibril formation in the pancreatic tissues of a subject, to shift an equilibrium which exists between smaller and larger MW species of IAPP amyloid toward the smaller species of IAPP oligomers and fibrils, or to reduce the level of IAPP in the tissues below the threshold amount by inducing LL-37 production in the pancreatic tissues. In some embodiments, these pharmaceutically acceptable compositions preferably include a mixture of at least four more preferably at least five, and most preferably at least six materials (preferably active materials) selected from the group consisting of phenylbutyrate, bexarotene, curcumin, resveratrol, retinol, cholecalciferol, fatty acids, and pharmaceutically acceptable salts thereof. In other embodiments, the pharmaceutically acceptable compositions disclosed herein preferably include a mixture of at least four more preferably at least five, and most preferably at least six materials (preferably active materials) selected from the group consisting of phenylbutyrate, bexarotene, curcumin, resveratrol, retinol, cholecalciferol, docosahexaenoic acid, caprylic acid, capric acid, lauric acid, and pharmaceutically acceptable salts thereof. In still other embodiments, the pharmaceutical composition may comprise the Bacille Calmette-Guerin (BCG) vaccine.

These pharmaceutical compositions may utilize one or more active ingredients (and will preferably utilize multiple active ingredients, as noted above) which may be dissolved, suspended or disposed in various media. Such media may include, for example, various liquid, solid or multistate media such as, for example, emulsions, gels or creams. Such media may include liquid media, which may be hydrophobic or may comprise one or more triglycerides or oils. Such media may include, but is not limited to, vegetable oils, fish oils, animal fats, hydrogenated vegetable oils, partially hydrogenated vegetable oils, synthetic triglycerides, modified triglycerides, fractionated triglycerides, and mixtures thereof. Triglycerides used in these pharmaceutical compositions may include those selected from the group consisting of almond oil; babassu oil; borage oil; blackcurrant seed oil; black seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; and mixtures thereof. The use of coconut oil or MCT (medium chain triglyceride) oil is especially preferred.

Various fatty acids may be utilized in the pharmaceutical compositions disclosed herein. These include, without limitation, both long and short chain fatty acids. Examples of such fatty acids include, but are not limited to, docosahexaenoic acid, caprylic acid, capric acid, lauric acid, butyric acid, and pharmaceutically acceptable salts thereof.

The pharmaceutical compositions disclosed herein may be applied in various manners. Thus, for example, these compositions may be applied as oral, transdermal, transmucosal, intravenous or injected treatments, or via cell-based drug delivery systems. Moreover, these compositions may be applied in a single dose, multi-dose or controlled release fashion.

The pharmaceutical compositions disclosed herein may be manufactured as tablets, liquids, gels, foams, ointments or powders. In some embodiments, these compositions may be applied as microparticles or nanoparticles.

Various counterions may be utilized in forming pharmaceutically acceptable salts of the materials disclosed herein. One skilled in the art will appreciate that the specific choice of counterion may be dictated by various considerations. However, the use of sodium and hydrochloride salts may be preferred in some applications.

In some embodiments of the systems and methodologies disclosed herein, rather than upregulating cathelicidin gene expression in the subject, peptoid mimics or peptidomimetics of LL-37 or its fragments may be utilized or administered to the subject for similar purposes. These may include modified peptides, structural mimetics (including peptidic foldamers), and mechanistic mimetics.

In some embodiments, a plurality of mimetics may be utilized, which may or may not be connected by a linker moiety. For example, in one such embodiment, two mimetics (peptidomimetics) of LL-37 binding regions may be connected by a flexible peptoid linker such as, for example, an oligo-N-methoxyethylglycine or oligo-Nmeg. However, various other linker moieties may also be utilized including, but not limited to, peptides, PEG, peptoids, 7-aminoheptanoic acid (AHA), alkyl linkers, and linkers containing disulfide or triazole-moieties. Preferably, the peptidomimetics include a first mimetic of the N-terminal sequence LL-37(1-15) of LL-37, and a second mimetic of the C-terminal sequence LL-37(18-34) of LL-37. More preferably, the peptidomimetics include a first mimetic of the LL-37(6-10) or FRKSK of LL-37 at the N-terminus, and a second mimetic of the LL-37(25-27) or KDF region within the C-terminal part of LL-37.

### Exemplary Embodiments

The following embodiments are provided to further disclose and elucidate the current disclosure. These embodiments are of an exemplary nature and are not meant to limit the scope of the disclosure.

### Peptides and Peptide Synthesis

IAPP was synthesized using Fmoc-solid phase synthesis strategy on Rink resin, oxidized with air and purified with reverse phase (RP) HPLC as previously described. IAPP stock solutions were made by dissolving the peptide in 1,1,3,3,3,3-hexafluoro-2-isopropanol (HFIP) (at 4° C.) and filtering the solution as described; IAPP concentration was determined by UV spectroscopy. N^{h}-amino-terminal fluorescein labeled IAPP (Fluos-IAPP) was synthesized, purified (MALDI-TOF MS: found MH+, 4261.2; calculated 4262.2), and handled as previously described. LL-37 was purchased from BACHEM and from AnaSpec; its stock solutions were made by dissolving it in HFIP (at 4° C.); its concentration was determined by its weight and the BCA assay. Scrambled LL-37 (scrLL-37) and N-amino-terminal fluorescein labeled LL-37 (FAM-LL-37) were purchased from Anaspec; of note, a 6-aminohexanoic acid spacer was included between the fluorescein (FAM) moiety and the N-terminus of LL-37 in FAM-LL-37. Synthetic glucagon (control for dot blot assays) was from BACHEM. The LL-37 partial segments LL-37(1-14) and LL-37(15-37) were synthesized on Wang-resin using previously established Fmoc-SPPS protocols both manually and by a CS336X peptide synthesizer (CS Bio). Briefly, couplings were performed (twice or 3 times) using standard Fmoc-protected amino acids (3-fold molar excess) and as coupling reagents N,N,N′,N′-tetramethyl-O-(1H-benzotriazol-1-yl)uronium hexafluorophosphate (HBTU) or 2-(7-aza-1H-benzotriazole-1-yl)-1,1,3,3-tetramethyluronium hexafluorophosphate (HATU) (3-fold molar excess) for selected couplings, and N,N-diisopropylethylamine (DIEA) (4.5 molar excess) in N,N-dimethylformamide (DMF). In the case of LL-37(1-14), most couplings were performed twice using HBTU as coupling agent except for Lys10, Glu11, and Lys12 for which HATU was used for the first coupling; in addition, 3 couplings were performed for Phe5, Phe6, and Arg7 using HATU for the first two of them. In the case of LL-37(15-37), most couplings were repeated three times using HATU for the first one and HBTU for the following ones; double couplings were performed only within LL-37(15-19), and for residues Lys25, Asp26, Pro33, and Arg34. Cleavages of the two LL-37 segments from the resin were carried out by trifluoroacetic acid/water (95/5, v/v) (3 h). Their RP-HPLC purifications were performed using a Nucleosil 100 C18 (250 mm×8 mm; particle size, 7 μm) column as described. Peptide purity (including the purity of commercially obtained LL-37, scrLL-37 and FAM-LL-37) was verified by MALDI-TOF MS. In the case of LL-37(1-14): found MH+, 1638.3 (calculated, 1638.9); in the case of LL-37(15-37), found MH+, 2873.9 (calculated, 2873.6). Stocks of LL-37(1-14) and LL-37(15-37) were made in HFIP (4° C.) and their concentrations were determined by their weight and confirmed by the BCA assay.

### Thioflavin T (ThT) Binding Assays

The effects of LL-37 and the other peptides on kinetics of IAPP fibrillogenesis were investigated by using the thioflavin T (ThT) binding assay using a previously established protocol. Briefly, IAPP alone (16.5 μM) and its mixtures with LL-37 and the other peptides were incubated in ThT assay buffer (aqueous 50 mM sodium phosphate buffer, pH 7.4, containing 100 mM NaCl and 0.5% HFIP) at the indicated molar ratios (20° C.) (non-stirring conditions). Of note, an incubation of LL-37 alone was also included. At the indicated time points aliquots were mixed with the ThT solution consisting of 20 μM ThT in 0.05 M glycine/NaOH (pH 8.5) and binding was determined by measuring fluorescence emission at 486 nm upon excitation at 450 nm using a Multilabel reader VictorX3 (Perkin Elmer Life Sciences). For studying the effect of LL-37 on nucleation of IAPP fibrillogenesis, i.e. following seeding with preformed IAPP fibrils (fIAPP) (10%), incubations of IAPP (16.5 μM) and its mixtures with LL-37 (1/1) were performed as above at room temperature. An aliquot of a solution consisting mostly of IAPP fibrils (fIAPP) based on ThT binding and TEM (FIGS. 18-19) (7 days aged IAPP (16.5 μM) made as above) was added to the incubations resulting in a final seed (fIAPP) concentration of 1.65 μM. Incubations of (unseeded) IAPP alone (16.5 μM) and of 10% fIAPP alone were included as controls. ThT binding was determined at the indicated time points as above. To study the effect of LL-37-treated IAPP fibrils (LL-37-treated fIAPP) on kinetics of IAPP fibrillogenesis in comparison to the seeding effect of fIAPP, an aliquot of a solution consisting mostly of fIAPP (16.5 μM fIAPP, 7 days aged; see above) was added to solid LL-37 (10-fold molar excess) and the mixture was incubated for 24 h yielding “LL-37-treated fIAPP” (FIG. 3b). Of note, binding of LL-37 to fIAPP was confirmed by a dot blot (DB) assay (FIG. 2b and data not shown); in addition, a DB assay was also applied to confirm that the same amounts of fIAPP were present in the aliquots of fIAPP and the LL-37-treated fIAPP used for the seeding assays (data not shown). An aliquot of the fIAPP containing solution (treated in the same way as the LL-37-treated aliquot but w/o LL-37) was used to determine the seeding effect of fIAPP. Solutions containing (unseeded) IAPP alone (16.5 μM), IAPP seeded with fIAPP (10%), and IAPP seeded with LL-37-treated fIAPP (10%) were made in ThT assay buffer and kinetics of fibrillogenesis were determined by the ThT binding assay as described above.

### Assessment of Cell Damage by the MTT Reduction Assay

Effects of LL-37 and the other peptides on formation of β-cell damaging IAPP assemblies were studied in the rat insulinoma cell line RIN5fm using the peptide solutions applied for the ThT binding assays as previously described. Briefly, RIN5fm cells were cultured and plated in 96-well plates as described. Solutions of IAPP alone and its mixtures with peptides were aged in ThT assay buffer as described under “ThT binding assays”. At the indicated incubation time points (24 h or 7 days) aliquots were diluted with cell culture medium and added to the cells. Following incubation with the cells for ˜20 h (37° C., humified atmosphere containing 5% CO2), cell damage was verified by the MTT reduction assay. For the determination of the IC50 of the inhibitory effect of LL-37 on formation of cytotoxic IAPP aggregates, 24 h aged IAPP (100 nM) alone and its mixtures with various amounts of LL-37 (made as under ThT binding assay) were added to the cells and cell viability was determined by the MTT reduction assay as above.

### Transmission Electron Microscopy (TEM)

TEM samples were prepared applying 10 μl aliquots of the solutions used in the ThT binding and MTT assays on carbon-coated grids at the indicated time points. The grids were washed using ddH2O and stained with aqueous 2% (w/v) uranyl acetate solution. Examination of the grids was done with a JEOL 1400 Plus electron microscope at 120 kV.

### Far-UV CD Spectroscopy

Far-UV CD studies were performed using a Jasco 715 spectropolarimeter. Spectra were recorded at room temperature between 195 and 250 nm, at 0.1 nm intervals, and with a response time of 1 second. Each spectrum is an average of 3 spectra. All CD studies were performed in aqueous 10 mM sodium phosphate buffer (pH 7.4) containing 1% HFIP (CD assay buffer) at room temperature; this assay system has been earlier developed and found to be suitable for following kinetics of IAPP (5 μM) misfolding into β-sheets and amyloid fibrils alone or in the presence of inhibitors. Briefly, peptide stocks in HFIP were freshly made (4° C.), diluted with assay buffer (room temperature) at the indicated concentrations within the cuvette, and following gentle mixing spectra were measured immediately or at the indicated incubation time points. For the studies addressing the interactions between LL-37 or scrLL-37 and IAPP, peptide mixtures (1/1) were prepared in HFIP (4° C.) and diluted with assay buffer in the cuvette at the indicated concentrations (5 μM each) (room temperature); CD spectra were measured as above. Of note, CD studies on IAPP alone and LL-37 or scrLL-37 alone (from the same stocks; 5 μM) were also performed in parallel. The CD spectrum of IAPP at the incubation time point of 24 h (endpoint) was measured after gentle mixing to re-dissolve precipitated aggregates. The CD spectrum of the buffer was always subtracted from the CD spectra of the peptide solutions.

### Fluorescence Spectroscopic Titrations

Fluorescence spectroscopic titration studies were performed with a JASCO FP-6500 fluorescence spectrophotometer using a previously described experimental protocol. Briefly, excitation was at 492 nm and emission spectra were recorded between 500 and 600 nm. The apparent (app.) Kds of the interactions of IAPP with LL-37 and its segments LL-37(1-14) and LL-37(15-37) were quantified by titrating synthetic Na-amino-terminal fluorescein labeled IAPP (5 nM) with various amounts of each of the peptides. For all experiments, freshly made stocks of peptides and their fluorescently labeled analogs in HFIP were used. Measurements were performed in 10 mM sodium phosphate buffer (pH 7.4) containing 1% HFIP within 2-5 min following solution preparation at room temperature. Of note, under these conditions freshly made Fluos-IAPP (5 nM) solutions consist mostly of monomers. App. Kds were calculated using 1/1 binding models as previously described and are means (±SD) of three binding curves.

### Cross-Linking, NuPAGE, and Western Blot Analysis

Cross-linking studies were preformed using a previously developed assay system. Briefly, solutions of IAPP alone (30 μM) and its mixtures with LL-37 or scrLL-37 at the indicated molar ratios (IAPP/LL-37 at 1/1 or 1/0.1) were prepared in aqueous sodium phosphate buffer, pH 7.4, and incubated for 30 min at room temperature; of note, incubations of LL-37 alone (at the same concentrations as in its mixtures with IAPP) were made as well. Solutions were cross-linked using 25% aqueous glutaraldehyde (Sigma-Aldrich) and 10% aqueous trichloroacetic acid (TCA) was used to precipitate cross-linked peptides. Pellets were dissolved in reducing NuPAGE sample buffer, boiled for 5 min, and subjected to NuPAGE electrophoresis in 4-12% Bis-Tris gels with MES running buffer (Invitrogen). The same volume of each solution (same IAPP amount) was loaded in all lanes. Peptides were blotted using a XCell II Blot Module blotting system (Invitrogen). IAPP or LL-37 were detected using a polyclonal rabbit anti-IAPP antibody (Peninsula) or a monoclonal mouse anti-LL-37 antibody (Santa Cruz Biotechnology), respectively in combination with suitable peroxidase (POD)-coupled secondary antibodies (Pierce & Amersham) and the Super Signal West Dura Extended Duration Substrate (Pierce). Of note, previous studies provided evidence for the specificity of the cross-linking assay; in addition, no new bands were observed in IAPP-scrLL-37 (1/1) mixtures (data not shown).

### Dot Blot Analysis

IAPP monomers or IAPP fibrils (fIAPP) containing solutions (different amounts up to 40 μg) were spotted onto a nitrocellulose membrane. These solutions were prepared by incubating an IAPP solution (1 mg/ml) in ThT assay buffer for 0 h (“monomers”) or 24 h (“fibrils”); the presence of fibrils was confirmed by ThT binding and TEM (not shown). The membrane was washed with TBSn (20 mM Tris/HCl, 150 mM NaCl and 0.05% Tween-20), blocked with 5% milk in TBSn overnight at 10° C., and washed again with TBSn. Then, the membrane was incubated with N-terminal fluorescein labeled LL-37 (FAM-LL-37 from AnaSpec; see under “Peptides and peptide synthesis”) (200 nM) in ThT assay buffer containing 1% HFIP overnight at 10° C. Following washings with incubation buffer and TBSn, bound FAM-LL-37 was visualized with a LAS-4000mini instrument (Fujifilm). Of note, glucagon fibrils were spotted as well to control for the specificity of the observed strong binding of FAM-LL-37 to fIAPP (not shown). Glucagon fibrils were made by incubating glucagon in 10 mM HCl (2 μg/μl) (10 days) followed by neutralization with 10 mM NaOH; ThT binding and TEM confirmed fibril formation (not shown). The ThT buffer alone was also spotted to control for NSB. In addition, to control for the interference of fibril autofluorescence, a membrane containing spotted fIAPP which had been incubated in buffer alone w/o FAM-LL-37 was included in each assay; in general, fIAPP autofluorescence contributed up to 25% of the total amount of fluorescence observed in fIAPP bound to FAM-LL-37.

### Determination of LL-37 Binding Sites by Using Peptide Arrays

A peptide array consisting of LL-37 decamers covering the full length LL-37 sequence and positionally shifted by one residue was synthesized on a modified cellulose membrane support using stepwise SPOT synthesis protocols and a MultiPep RSi (Intavis) peptide synthesizer. Thereafter, peptides were immobilized on a glass slide according to the manufacturer's instructions followed by a blocking step using 1% BSA in TBSn for 4 h (room temperature). The glass slide with the peptide array was incubated with a solution of Fluos-IAPP (1 μM in TBSn containing 1% BSA) for ˜12 h at 10° C. followed by washing with TBSn. Visualization of bound Fluos-IAPP was performed with a LAS-4000mini instrument (Fujifilm).

### Sequence Alignment Using LALIGN

The sequence alignment of IAPP and LL-37 was done with the program LALIGN (Author: Bill Pearson; https://embnet.vital-it.ch/software/LALIGN_form.html). Of note, this program was previously used for the comparison of the Aβ and IAPP sequences to each other. A global alignment method was used with 3 reported sub-alignments; E-value threshold was set to 10.0, the scoring matrix used is BLOSUM50, opening gap penalty was set to −12 and extending gap penalty to −2 (default values). The LALIGN program implements the algorithm of Huang and Miller.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims.

1. A method for treating type 2 diabetes (T2D), comprising:
diagnosing a subject as suffering from T2D or as being pre-diabetic;
monitoring the response to glucose stimulation of at least one islet in the pancreas of the subject by quantitatively imaging glucose metabolism in vivo;
establishing a target range for the response to glucose stimulation of the at least one islet; and
upregulating cathelicidin gene expression in the subject until the monitored response to glucose stimulation is within the target range.

2. The method of claim 1, wherein monitoring the response to glucose stimulation includes determining the response to glucose stimulation of the at least one islet occurs in real time.

3. The method of claim 1, wherein monitoring the response to glucose stimulation includes quantitatively imaging real-time glucose metabolism from single islets in vivo.

4. The method of claim 1, wherein monitoring the response to glucose stimulation includes utilizing intrinsic autofluorescence in combination with multiphoton excitation microscopy.

5. The method of claim 1, wherein monitoring the response to glucose stimulation includes quantitatively imaging glucose metabolism in vivo to repeatedly measure the response of the at least one islet to glucose stimulation.

6. The method of claim 1, wherein monitoring the response to glucose stimulation further includes monitoring at least one parameter selected from the group consisting of islet function, proliferation, vasculature and macrophage infiltration.

7. The method of claim 1, wherein quantitatively imaging glucose metabolism includes NAD(P)H imaging.

8. The method of claim 7, wherein monitoring the response to glucose stimulation includes:
directly measuring glucose metabolism; and
correlating autofluorescence signals with downstream glucose-stimulated events.

9. (canceled)

10. The method of claim 7, wherein the NAD(P)H imaging uses an interdependence between NAD(P)H and intracellular Ca²⁺ to measure pancreatic β-cell function.

11. The method of claim 7, further comprising:
monitoring redox state and mitochondrial function by quantifying cellular NAD(P)H.

12. The method of claim 7, further comprising:
measuring cytoplasmic and mitochondrial NAD(P)H changes to resolve the spatiotemporal partitioning of glycolytic and oxidative metabolism.

13. The method of claim 1, wherein quantitatively imaging glucose metabolism in vivo includes the use of a β-Amyloid imaging probe.

14-16. (canceled)

17. The method of claim 13, wherein the β-Amyloid imaging probe is selected from the group consisting of IPBF, PQ-6, FPYBF-1, IMPY and AV-45 imaging probes.

18. The method of claim 17, wherein the β-Amyloid imaging probe is [¹²⁵I]IPBF.

19. The method of claim 1, wherein monitoring the response to glucose stimulation of at least one islet includes the use of positron emission tomography (PET) in combination with an amyloid imaging probe.

20. The method of claim 19, wherein the amyloid imaging probe is a ¹⁸F-labelled radiopharmaceutical.

21-24. (canceled)

25. The method of claim 1, wherein diagnosing a subject as being pre-diabetic includes administering to the subject a test selected from the group consisting of the Fasting Plasma Glucose (FPG) test, the Oral Glucose Tolerance Test (OGTT), and the Random Plasma Glucose test.

26. The method of claim 1, wherein upregulating cathelicidin gene expression in the subject includes administering to the subject a pharmaceutically acceptable composition which upregulates cathelicidin gene expression and wherein said mixture includes at least three materials selected from the group consisting of phenylbutyrate, bexarotene, curcumin, resveratrol, retinol, betacarotene, cholecalciferol, and pharmaceutically acceptable salts thereof.

27-44. (canceled)

45. A method for treating a subject, comprising:
monitoring levels of the cathelicidin peptide LL-37 in the blood of a subject and IAPP amyloid in pancreatic tissues of the subject; and
when the condition L/B<k is detected, where L is the level of LL-37 detected, B is the level of IAPP detected, and k is a predetermined threshold value, upregulating cathelicidin gene expression in the subject.

46-80. (canceled)

81. A method for treating a subject for type 2 diabetes (T2D), comprising:
diagnosing the subject as suffering from T2D; and
applying to the subject a pharmaceutically acceptable composition comprising a peptidomimetic of LL-37 or a portion thereof.

82-87. (canceled)


SEQUENCE LISTING (35 sequences)

SEQ ID NO: 1 - protein, 37 aa, homo sapiens [sequence omitted]

SEQ ID NO: 2 - protein, 37 aa, homo sapiens
LLGDFFRKSKEKIGKEFKRIVQRIKDFLRNLVPRTES

SEQ ID NO: 3 - protein, 14 aa, Homo sapiens
LLGDFFRKSKEKIG

SEQ ID NO: 4 - protein, 23 aa, Homo sapiens
KEFKRIVQRIKDFLRNLVPRTES

SEQ ID NO: 5 - protein, 37 aa, Homo sapiens
GLKLRFEFSKIKGEFLKTPEVRFRDIKLKDNRISVQR

SEQ ID NO: 6 - protein, 15 aa, Homo sapiens
LLGDFFRKSKEKIGK

SEQ ID NO: 7 - protein, 17 aa, Homo sapiens
KRIVQRIKDFLRNLVPR

SEQ ID NO: 8 - protein, 10 aa, Homo sapiens
LLGDFFRKSK

SEQ ID NO: 9 - protein, 10 aa, Homo sapiens
LGDFFRKSKE

SEQ ID NO: 10 - protein, 10 aa, Homo sapiens
GDFFRKSKEK

SEQ ID NO: 11 - protein, 10 aa, Homo sapiens
DFFRKSKEKI

SEQ ID NO: 12 - protein, 10 aa, Homo sapiens
FFRKSKEKIG

SEQ ID NO: 13 - protein, 10 aa, Homo sapiens
FRKSKEKIGK

SEQ ID NO: 14 - protein, 10 aa, Homo sapiens
RKSKEKIGKE

SEQ ID NO: 15 - protein, 10 aa, Homo sapiens
KSKEKIGKEF

SEQ ID NO: 16 - protein, 10 aa, Homo sapiens
SKEKIGKEFK

SEQ ID NO: 17 - protein, 10 aa, Homo sapiens
KEKIGKEFKR

SEQ ID NO: 18 - protein, 10 aa, Homo sapiens
EKIGKEFKRI

SEQ ID NO: 19 - protein, 10 aa, Homo sapiens
KIGKEFKRIV

SEQ ID NO: 20 - protein, 10 aa, Homo sapiens
IGKEFKRIVQ

SEQ ID NO: 21 - protein, 10 aa, Homo sapiens
GKEFKRIVQR

SEQ ID NO: 22 - protein, 10 aa, Homo sapiens
KEFKRIVQRI

SEQ ID NO: 23 - protein, 10 aa, Homo sapiens
EFKRIVQRIK

SEQ ID NO: 24 - protein, 10 aa, Homo sapiens
FKRIVQRIKD

SEQ ID NO: 25 - protein, 10 aa, Homo sapiens
KRIVQRIKDF

SEQ ID NO: 26 - protein, 10 aa, Homo sapiens
RIVQRIKDFL

SEQ ID NO: 27 - protein, 10 aa, Homo sapiens
IVQRIKDFLR

SEQ ID NO: 28 - protein, 10 aa, Homo sapiens
VQRIKDFLRN

SEQ ID NO: 29 - protein, 10 aa, Homo sapiens
QRIKDFLRNL

SEQ ID NO: 30 - protein, 10 aa, Homo sapiens
RIKDFLRNLV

SEQ ID NO: 31 - protein, 10 aa, Homo sapiens
IKDFLRNLVP

SEQ ID NO: 32 - protein, 10 aa, Homo sapiens
KDFLRNLVPR

SEQ ID NO: 33 - protein, 10 aa, Homo sapiens
DFLRNLVPRT

SEQ ID NO: 34 - protein, 10 aa, Homo sapiens
FLRNLVPRTE

SEQ ID NO: 35 - protein, 10 aa, Homo sapiens
LRNLVPRTES